(19) United States
(12) Patent Application Publication
(10) Pub. No.: US 2007/0083179 A1
(43) Pub. Date: Apr. 12, 2007

(54) Used tampon and condom personal disposal

(76) Inventor: Amy L. Fuentes, Centreville, VA (US)

Correspondence Address: Amy L. Fuentes, 6609 Peaceful Meadow Lane, Centreville, VA, 22120, US

(21) Appl. No.: 11/245,119

(22) Filed: Oct. 7, 2005

(51) Int. Cl. A61F 13/15; B65D 33/02; B65D 33/00; B65D 33/16; B65D 85/08; B65D 30/08; B65D 85/84

(52) U.S. Cl. 604/385.02; 604/385.13; 604/385.19; 604/359; 383/35; 383/109; 383/63; 383/84; 383/80; 383/95; 383/34; 206/524.1; 206/69

(57) ABSTRACT

This is a bag for disposal of used tampons or condoms in a sanitary, discrete manner.

### BACKGROUND

1. Field of Invention

In the past, women have either flushed or wrapped in toilet paper their used tampons. However, many places such as hospitals, doctor's offices, health clubs, boats, and homes and businesses on septic, have requested that women not flush used tampons down the toilet. This leaves the latter alternative of wrapping their used tampon in a cocoon of toilet paper. If the bathroom facilities do not have a bin in the stall, some women have put the wrapped used tampon in their purse to avoid the embarrassment or out of respect to the home or boat owner.

2. Prior Art

Proper disposal of used tampons is important for sanitary and discretionary reasons. Currently, there is little available for personal disposal. Many inventions have tried to solve this problem, however, they have not met all the needs necessary for doing so.

A search for prior art has uncovered the following related patents.

U.S. Pat. No. 2,656,769—Hultkrans, which describes producing a reusable pouch having a drawstring. Once a pouch is used, it is unhygienic to use it again.

U.S. Pat. No. 3,079,966—Kugler, which describes producing a particular bag configuration using a drawstring for closure. This type of closure is inefficient at best. Blood can come out of the bag and soil a woman's purse or pocket that she may be using to temporarily store the item.

U.S. Pat. No. 3,132,794—Frazier, which describes producing a foldable bag or receptacle that uses a drawstring for closure. Again, this closure is inefficient as stated above.

U.S. Pat. No. 3,920,179—Hall, which describes a disposable, flexible, partitionable bag with a drawstring to effect the partitioning. The bag has a rigid support and the patent describes placing a deodorant pill inside to reduce the unpleasant odors. Once again, the closing mechanism is inefficient. Also, having a rigid support prevents the user from discreetly carrying it in their pocket or small purse.

U.S. Pat. No. 4,036,363—Kugler, which describes the automatic filling of bags and the bags having a top opening and drawcord. Needless to say, this type of closure is ineffective.

U.S. Pat. No. 4,765,477—Froidh et al., which describes a wrapper that when taken off a clean tampon can be used for the disposal of a used one. Users typically remove the used tampon before opening the wrapper of a new one. Therefore, in order to use this invention, the new tampon would have to be removed from the wrapper and placed on a potentially unsanitary surface such as a sink or top of a toilet, while the used tampon is removed and disposed of. If the used tampon is taken out before the wrapper is opened, the bloody tampon would have to be placed on a surface and thus would contaminate it. This is awkward for someone to use.

U.S. Pat. No. 4,838,327—which describes a bag assembly with an insert that can be withdrawn to a certain point. It is not discussed what this bag is made of, however, if it is made out of cardboard or other material that is absorbent, a bloody mess would be the result. This is also cumbersome and complicated to use and there is the possibility of getting menstrual fluid on the outside bag when the insert is lowered into the outer pouch. Finally, it is more difficult and expensive to manufacture with the latch mechanism and insert.

U.S. Pat. No. 4,996,727—Wyatt, which describes a three layered bag with a handle. This device is clumsy to use and while fitting the tampon into the bag could result in residue getting onto the outside of the bag.

U.S. Pat. No. 5,133,457—which describes using the existing wrapper of a tampon for disposal of the applicator after the tampon has been inserted. This invention would not make a viable option for the disposal of the used tampon due to the wrapper being made from material that is thin, easily ripped and having no way to close the wrapper once the object is inserted. Also, because the opening is the size of the applicator and the used tampon has increased in size due to the absorption of blood, there is a good possibility of getting menstrual fluid on the outside of the wrapper.

U.S. Pat. No. 5,193,684—McDonald, which describes a Tampon disposal Unit. This invention is put over the opening and the tampon is withdrawn directly into the bag as it unfolds longitudinally. This complicated design makes it confusing as to the proper way to use it. There is great difficulty in using this device given the many steps needed to use it and preventing menstrual fluid from getting on the outside would be almost impossible.

U.S. Pat. No. 5,740,554—Reed, which describes a mitten device used to remove the tampon and wrap over it. This is awkward to use and has a possibility of tearing thus producing a hole in which the blood can leak onto the user's hand.

U.S. Pat. No. 6,402,727—which describes a flat disposal cover with adhesive tapes. This invention, although easy to use, does not securely house a used tampon and prevent the leakage of the blood since it is sealed in select places leaving potential openings.

### SUMMARY OF INVENTION

The creation of a non-permeable, sealable pouch for women to place used tampons to prevent the clogging of pipes or the excessive use of toilet paper to wrap the used tampon. This pouch allows women to dispose of used tampons hygienically by containing the blood in a non-permeable, closable bag thus preventing the potential spread of diseases. In addition, the bag can be used to dispose of condoms for men. This provides a hygienic and non-embarrassing alternative to placing them in a trash receptacle.

### DETAILED DESCRIPTION

The inside bag is large enough to hold a used condom or tampon and applicator with the outer bag concealing the contents of the inside bag. Since the tampon increases in size as it absorbs the fluid, the bag is big enough to accommodate the used tampon and applicator used to insert a new tampon. The opening is large enough to easily fit a used tampon and applicator without the possibility of getting residue on the outside of the pouch. When not in use the bag lays flat in order to be easily stored in small places like a pocket or purse. After it has the used items enclosed, it is no bigger than the items being put into it. This enables the user to store the bag in a pocket or small purse until it can be adequately disposed.

The shape may include but not limited to rectangle, square, circle, triangle, octagon, hexagon, cylinder, cone, tube, and box. As long as the shape allows the containment of the used tampon and applicator, any shape will work.

The material is non-permeable and may include but not limited to thin pliable plastic (such as a sandwich or freezer bag); thick, hard, plastic (such as an unbreakable jar); medium plastic (such as is used in toothpaste tubes); aluminum; and PVC. This allows the blood to be contained and not leak. Thus, avoiding any embarrassing moments for the person carrying the used items.

The material may be any color. It may or may not have an outer cover comprising of a bag, to ensure the contents are not seen should a transparent substance such as clear plastic be used. The material used for the cover may include but not be limited to fabric, opague plastic, heavy paper or cardboard. The bag can be made in a variety of colors and patterns to match the users' purse or pocket. If an outer container is used, this container can be made in a variety of colors or patterns for the same purpose.

The pouch may or may not be scented or have a deodorant inside. This can be a pad, powder or anything else known to deodorize. Although the bag will be sealed, some people may want a nice smelling bag to carry around. This scent may include but not limited to citrus, perfume, or baby powder.

The closure means may include but not limited to a flap, button, or series of buttons, snap, or series of snaps, zipper, rib and groove mechanism wherein rib part is a horizontal interlockable part with the opposing groove profile—when the rib and groove profiles are interlocked, an audible clicking sound and/or bumpy feel perceptible to the touch is imparted, clip, buckle, clasp, hook and eye, hook and loop fastener, resealable tape (such as is used on post-it-notes), latch, screw or snap on cap, flip top cap, and hinges attached to a thin flat surface that when squeezed will open the pouch (such as was common in change pouches). Any of these closure means or other may be used as long as the bag is completely sealed to prevent any leakage of blood or smell.

What is claimed is:

1. An inner bag with a shape (consisting) comprising of:
sealed sides and bottom with the top portion open, the opening is stiff enough that when the sides are squeezed together the bag easily opens wide enough to insert a used tampon and applicator;
closure means associated with said bag for closing the bag's open end, the closure is complete thereby assuring that no seepage of the contents will occur;
an outer bag to conceal the contents of the inner bag with a shape comprising of:
sealed sides and bottom big enough to hold the inner bag with the top portion open;
closure means associated with said outer bag for closing the bag's open end.

2. The bag of claim 1 wherein said bag is made of a non-permeable substance.

3. The bag of claim 1 wherein said bag is opaque; it can be any color or pattern of colors.

4. The bag of claim 1 wherein said bag is clear with an outer bag that is opaque; it can be any color or pattern of colors.

5. The outer bag of claim 4 wherein said outer bag is made with fabric.

6. The outer bag of claim 4 wherein said outer bag is made with opaque plastic.

7. The outer bag of claim 4 wherein said outer bag is made with cardboard.

8. The outer bag of claim 4 wherein said outer bag is made with paper.

9. The bag of claim 2 wherein said bag is thin pliable plastic (such as) including a freezer bag.

10. The bag of claim 2 wherein said bag is a thicker, hard plastic (such as) including an unbreakable jar.

11. The bag of claim 2 wherein said bag is a medium plastic (such as) including used in toothpaste tubes.

12. The bag of claim 1 wherein said closure means is a (Ziplock) rib and groove mechanism.

13. The bag of claim 1 wherein said closure means is a zipper mechanism.

14. The bag of claim 1 wherein said closure means is a secured flap.

15. The bag of claim 1 wherein said closure means is a snap or a series of snaps.

16. The bag of claim 1 wherein said closure means is a latch.

17. The bag of claim 1 wherein said closure means is a screw or snap on cap.

18. The bag of claim 1 wherein said closure means is a flip top cap.

19. The bag of claim 1 wherein said closure means is a button or a series of buttons.

20. The bag of claim 1 wherein said closure means is (Velcro) hook and loop fastener.

21. The bag of claim 1 wherein said closure means is resealable tape.

22. The bag of claim 1 wherein said closure means is two thin metal flat pieces on the front and back that are attached with hinges that when the hinges are pushed together the metal flexes and opens the bag and when released will remain together and flat to close the bag.

23. The bag of claim 14 wherein said flap is secured with tape that is already on the flap that when the paper covering the tape is removed provides a sticky substance to close the opening.

24. The bag of claim 14 wherein said flap is secured with snaps.

25. The bag of claim 14 wherein said flap is secured with a latch.

26. The bag of claim 14 wherein said flap is secured with (Velcro) hook and loop fastener.

27. The bag of claim 14 wherein said flap is secured with hook and eye.

28. The bag of claim 14 wherein said flap is secured with a clasp.

29. The bag of claim 14 wherein said flap is secured with a clip.

30. The bag of claim 14 wherein said flap is secured with a buckle.

31. The bag of claim 1 wherein said bag('s) has a shape in the form of a rectangle.

32. The bag of claim 1 wherein said bag('s) has a shape in the form of a square.

33. The bag of claim 1 wherein said bag('s) has a shape in the form of a circle.

34. The bag of claim 1 wherein said bag('s) has a shape in the form of a triangle.

35. The bag of claim 1 wherein said bag('s) has a shape in the form of an octagon.

36. The bag of claim 1 wherein said bag('s) has a shape in the form of a hexagon.

37. The bag of claim 1 wherein said bag('s) has a shape in the form of a cylinder.

38. The bag of claim 1 wherein said bag('s) has a shape in the form of a cone.

39. The bag of claim 1 wherein said bag('s) has a shape in the form of a tube.

40. The bag of claim 1 wherein said bag('s) has a shape in the form of a box.

41. The bag of claim 1 wherein said bag contains a scent or perfume.

42. The bag of claim 1 wherein said bag contains a deodorant.

43. The bag of claim 1 wherein said bag contains a powder.

44. The bag of claim 1 that is used for disposing of used tampons and applicators.

45. The bag of claim 1 that is used for the disposing of used condoms.

46. The bag of claim 41 wherein said bag contains a lemon or citrus smell.

47. The bag of claim 41 wherein said bag contains a baby powder smell.

48. The bag of claim 1 has an opening big enough to hold the used tampon and applicator without getting any blood on the outside of the bag.
